(19) United States
(12) Patent Application Publication
(10) Pub. No.: US 2026/0272959 A1
(43) Pub. Date: Sep. 17, 2026

(54) NOVEL PHOSPHATE COMPOUNDS AND PROCESS FOR PREPARATION THEREOF

(71) Applicants: Rajaram SAMANT, Thane West (IN); Manoj TONGRA, Jaipur (IN)

(72) Inventors: Rajaram SAMANT, Thane West (IN); Manoj TONGRA, Jaipur (IN)

(21) Appl. No.: 19/167,239

(22) PCT Filed: Mar. 19, 2024
(86) PCT No.: PCT/IN2024/050283

§ 371 Date: Sep. 19, 2025

(30) Foreign Application Priority Data
Mar. 21, 2023 (IN) 202321019142

(51) Int. Cl. A61K 31/6615; C07F 9/09

(52) U.S. Cl. CPC A61K 31/6615; C07F 9/098

(57) ABSTRACT

The present invention relates to novel phosphate compounds of Formula I for treating central nervous system (CNS) disorders. Further, the present invention provides an improved process to obtain compounds of Formula (I) with high yield and purity. The novel phosphate compounds of Formula-I are useful in the treatment of neurological, neuropsychiatric, metabolic and neurodevelopmental disorders.

### TECHNICAL FIELD

The present invention relates to novel phosphate compounds of Formula I for treating central nervous system (CNS) disorders.

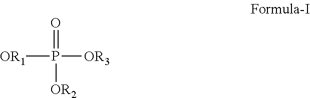

Particularly, the present invention relates to improved process to obtain phosphate compounds of Formula-I with good yield and purity. Further, the novel phosphate compounds of Formula-I are useful in the treatment of neurological, neuropsychiatric, metabolic and neurodevelopmental disorders.

### BACKGROUND AND PRIOR ART

Phosphorus belongs to one of the most essential elements of life and is extensively distributed in nature. It is an essential mineral found in every cell of the human body. Like calcium, sodium, magnesium, potassium, chloride, and sulfur, it is a macro-mineral.

Phosphorus is a mineral that naturally occurs in many foods. It is a key element of bones, teeth, and cell membranes. It helps to activate enzymes, and keeps blood pH within a normal range. Phosphorus regulates the normal function of nerves and muscles, including the heart, and is also a building block of human genes, as it makes up DNA, RNA, and ATP, the body's major source of energy. Phosphorus-containing compounds are involved in vital processes or functions ranging from biochemistry, biogeochemistry, ecology, agriculture, to industry.

Phosphates are medicinal salts of phosphorus. Phosphates are used as dietary supplements for patients who are unable to get enough phosphorus in their regular diet, usually because of certain illnesses or diseases. Phosphate is the drug form (salt) of phosphorus which is also called Phosphate ion or Orthophosphate. It is a trivalent inorganic anion and a conjugate base of hydrogen phosphate. Some phosphates are used to make medicines, which helps to treat certain urinary tract infections.

The main purpose of phosphorus is to build and maintain bones and teeth. It also plays a major role in the formation of DNA and RNA (the genetic building blocks of the body). Additionally, phosphorus plays a key role in metabolism (the conversion of calories and oxygen to energy), muscle contraction, heart rhythm, and the transmission of nerve signals. US10610469B2 relates to therapeutic calcium phosphate particles in use for aesthetic or cosmetic medicine, and methods of manufacture and use.

WO2007047378 directs to the dihydrogen phosphate salt of a pharmacologically active prostaglandin D2 receptor antagonist for treating, allergic disease, systemic mastocytosis, disorders accompanied by systemic mast cell activation, anaphylaxis shock, bronchoconstriction, bronchitis, urticaria, eczema, diseases accompanied by itch, diseases.

US9938270 B2 relates to crystalline and amorphous forms of 6-fluoro-2-[4-(pyridin-2-yl) but-3-yn-1-yl]imidazo[1,2-a]pyridine mono-phosphate salt and methods of making thereof. The invention compounds are modulators of mGlu5 receptors which are useful for the treatment or prevention of central nervous system disorders as well as other disorders modulated by mGlu5 receptors.

US10137061 B2 provides dental fillers and/or compositions that include a phosphate salt.

WO2020255978 relates to a novel phosphate ester having a pyrrolopyrimidine skeleton having antitumor effect.

US20070042999A discloses phosphatidyl derivative of opioids, steroid hormones, thyroid hormones, anaesthetics or chemotherapeutic agents having a phenolic, primary alcohol, secondary alcohol or tertiary hydroxyl group.

Inorganic phosphate (Pi) plays a critical role in skeletal development, mineral metabolism, and diverse cellular functions involving intermediary metabolism and energy-transfer mechanisms. The phosphate-calcium tandem contributes to the development and proper functioning of the organism. [Metabolites 2023, 13 (7), 860]

Phosphorus participates in practically all physiological chemical reactions. It is important for the proper functioning of the kidney, for the smooth functioning of the heart, for transmitting nerve impulses. Phosphorus is particularly important for the development of the nervous system and mental functions in children, as well as for the normal functioning of the nervous system. Phospholipids, phosphorus compounds, are necessary for the formation of a myelin sheath that protects nerve fibers.

Phosphorus is needed for the growth, maintenance, and repair of all tissues and cells. It is also needed to help balance and use other vitamins and minerals, including vitamin D, iodine, magnesium, and zinc. Phosphate-containing units are important building-blocks for nucleotides, which are the basic structures of deoxyribonucleic acid (DNA) and ribonucleic acid (RNA).

Phosphorus-containing drugs belong to an important class of therapeutic agents and are widely applied in daily clinical practices. Structurally, the phosphorus-containing drugs can be classified into phosphotriesters, phosphonates, phosphinates, phosphine oxides, phosphoric amides, bisphosphonates, phosphoric anhydrides, and others. Functionally, phosphorus-containing drugs are often designed as prodrugs with improved selectivity and bioavailability, reduced side effects and toxicity, or biomolecule analogues with endogenous materials and antagonistic endoenzyme supplements.

Most common applications are consistently developed due to their potential advantages in medicine or pharmaceutical. It is certain that phosphorus-containing drugs will continue to be an important class of valuable therapeutic agents in clinical applications. Considering use of known phosphate lipid salts and limitation thereof, currently there is need of therapeutically effective stable phosphate compounds with good bioavailability.

By knowing the current need of phosphorous containing compounds, the inventors of the present invention have developed therapeutically significant novel biochemical entity of phosphate of Formula-I in stable form.

### OBJECTIVE OF THE INVENTION

The primary object of the present invention is to provide novel therapeutical effective phosphate compounds of Formula-I.

Another object of the present invention is to provide novel phosphate compounds with distinct function groups.

Yet another object of the present invention is to provide therapeutically effective novel phosphate compounds.

Another object of the present invention is to provide novel class of phosphorus-containing entities which are useful in clinical applications.

Yet another objective of the invention is to provide phosphate compounds with improved selectivity and bioavailability, reduced side effects and toxicity.

### SUMMARY OF THE INVENTION

To meet the above objects, the inventors of the instant invention carried out thorough experiments to establish significant effects of the active ingredients or minerals or inorganic acid or esters or nutrients or metabolites or drugs or precursors or oxides that ameliorate therapeutic efficacy in the treatment of central nervous system related disorders.

In a particular aspect, the present invention provides novel phosphorus containing biochemical entities.

In another aspect, the present invention relates to novel therapeutically active phosphate compounds wherein the P—O anion linked to active cation.

In yet another aspect, the present invention provides novel phosphate compounds of Formula I for treating CNS disorders.

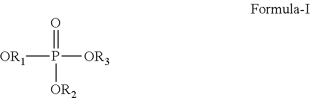

In a further aspect, the present invention provides pharmaceutical composition comprising novel phosphate compounds along with pharmaceutically acceptable excipients/carriers useful for treating CNS disorders wherein the composition containing 0.1-2000 mg of active phosphate moiety.

### BRIEF DESCRIPTION OF FIGURES

FIG. 1 illustrates FIGS. 1(a), 1(b) and 1(c) illustrates H1 NMR DMSO (400 MHz, CD3OD) of 1,3,4,9,10,10a-hexahydro-6-methoxy-11-methyl-2H-10,4alpha-iminoethanophenanthren 2-(trimethylammonio)ethyl hydrogen phosphate.

FIG. 2 illustrates 2 (a), 2 (b) illustrates H1 NMR DMSO (400 MHz, CD3OD) of 4-guanidinobutan-1-aminium 2-(trimethylammonio)ethyl hydrogen phosphate.

FIG. 3 illustrates HPLC chromatogram of (4-Aminobutyl) guanidine assay in 4-guanidinobutan-1-aminium 2-(trimethylammonio)ethyl hydrogen phosphate.

FIG. 4 illustrates HPLC chromatogram of 1,3,4,9,10,10a-hexahydro-6-methoxy-11-methyl-2H-10,4alpha-iminoethanophenanthren assay in 1,3,4,9,10,10a-hexahydro-6-methoxy-11-methyl-2H-10,4alpha-iminoethanophenanthren 2-(trimethylammonio)ethyl hydrogen phosphate.

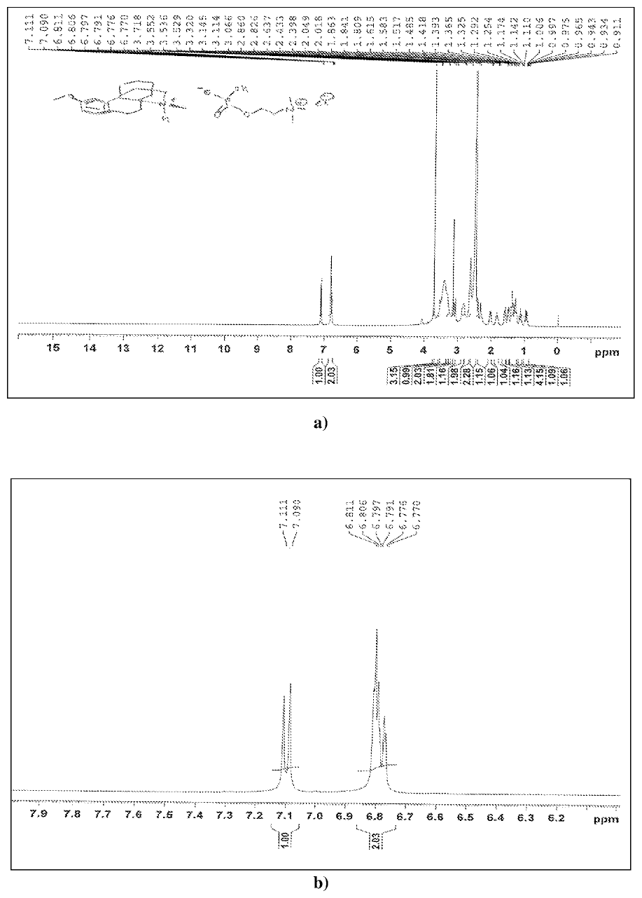

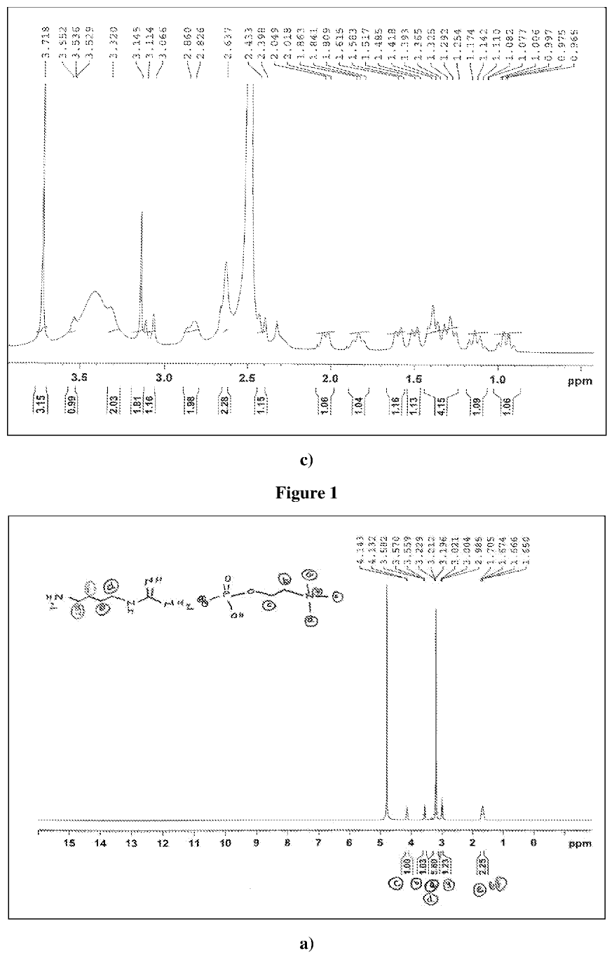

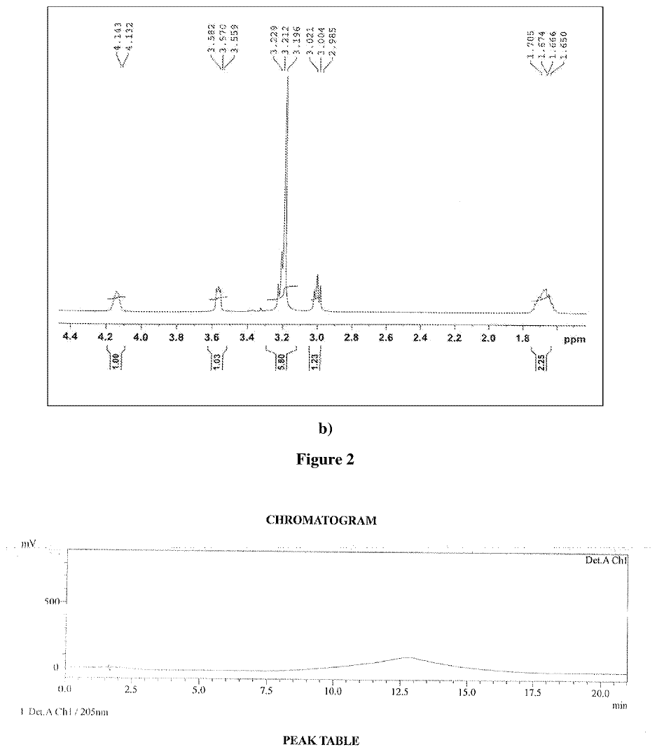

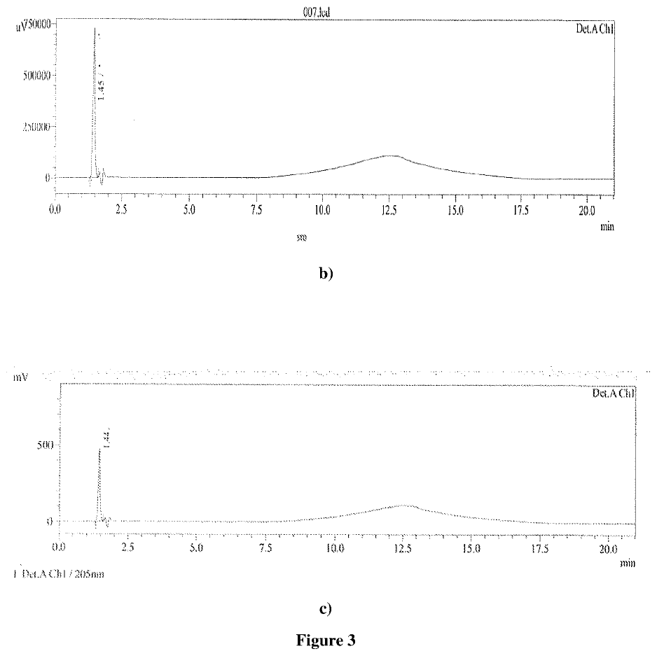

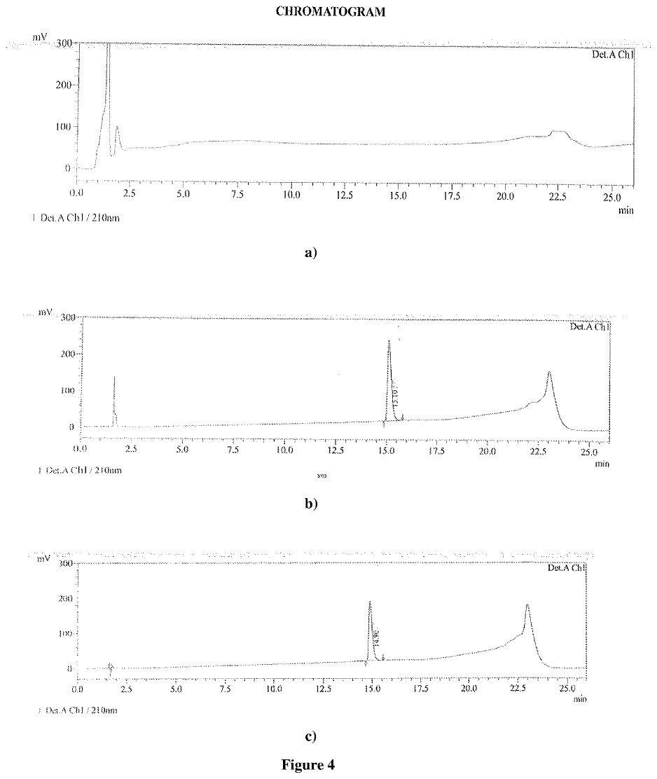

### DETAILED DESCRIPTION OF THE INVENTION

The invention will now be described in detail in connection with certain preferred and optional embodiments, so that various aspects thereof may be more fully interpreted and comprehended. However, any skilled person or artisan will appreciate the extent to which such embodiments could be generalized in practice.

It is further to be understood that all terminology used herein is for the purpose of describing particular embodiment only and is not intended to be limiting in any manner or scope. Unless defined otherwise, all technical and scientific expressions used herein have the same meaning as commonly understood by one of ordinary skill in the art to which embodiments of the invention pertain.

In describing and claiming the embodiments of the present invention, the following terminology will be used in accordance with the definitions set out below which are known in the state of art.

The singular forms “a,” “an,” and “the” include plural reference unless the context clearly dictates otherwise. Also, the term ‘composition’ does not limit the scope of the invention for multiple compositions that can be illustrated for best mode of the invention.

The term “pharmaceutically/nutraceutically acceptable salt,” as use herein, represents those salts which are within the scope of sound medical judgment, suitable for use in contact with the tissues of humans and animals without undue toxicity, irritation, allergic response and the like and are commensurate with a reasonable benefit/risk ratio.

Particularly the term “pharmaceutically acceptable salts” refers to the relatively non-toxic, inorganic and organic acid addition salts of compounds, amino acid salt, sugar-based salt, alkali or alkaline earth metal salts, as well as solvates, co-crystals, polymorphs and the like of the salts.

All modifications and substitutions that come within the meaning of the description and the range of their legal equivalents are to be embraced within their scope. A description using the transition “comprising” allows the inclusion of other elements to be within the scope of the invention.

In another preferred embodiment, the invention provides novel phosphate compounds of Formula I for treating CNS disorders.

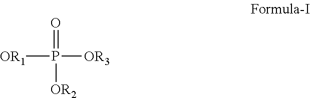

wherein R₁, R₂ and R₃ is similar or independently selected from the group consisting of hydrogen, hydroxyl, halogen, (C₁-C₆)alkyl, (C₂-C₈)alkylene, (C₂-C₈)alkyne, trifluoroalkyl, (C₃-C₈) cycloalkyl, (C₁-C₆)alkoxy, (C₆-C₁₀) aryl; heterocyclic groups such as substituted or unsubstituted pyrrole, pyridine, indole, quinoline; or
polyamines such as guanidine; or quaternary ammonium; or polycyclic aromatic hydrocarbon such as phenanthrene, anthracene; or nucleosides such as adenosine, guanosine, inosine
or
monovalent metal ion selected from the group Li⁺, Na⁺, K⁺; divalent metal ion selected from the group Ca²⁺, Mg²⁺, Zn²⁺ or combination thereof;

In yet another embodiment, the present invention relates to novel phosphate compounds of Formula I for treating CNS disorders.

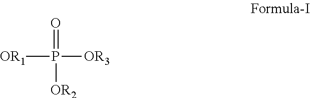

wherein R₁, R₂ and R₃ is similar or independently selected from the group consisting of hydrogen, hydroxyl, halogen, (C₁-C₆)alkyl; or groups like

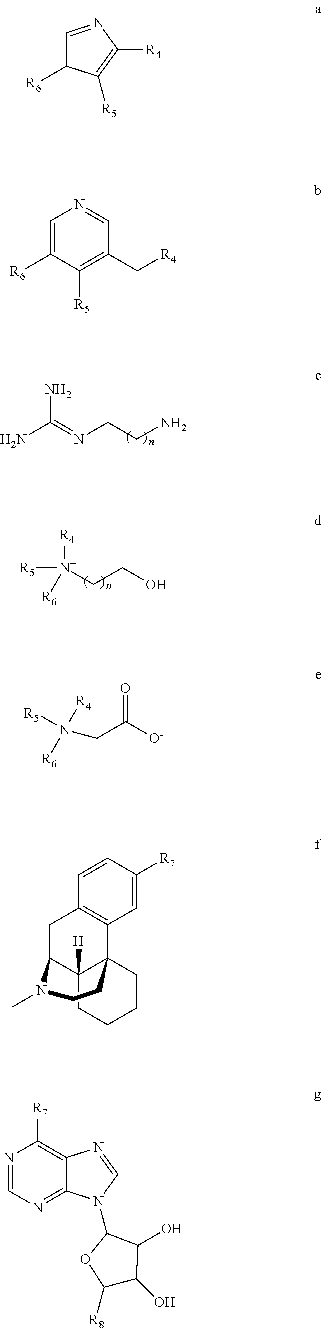

wherein R₄, R₅, R₆, R₇ and R₅ are similar or independently selected from the group consisting of hydrogen, hydroxyl, halogen, (C₁-C₆)alkyl, (C₂-C₈)alkylene, (C₂-C₈)alkyne, (C₁-C₆) alkoxy, (C₁ C₆)trifluoroalkyl, (C₃-C₈) cycloalkyl, (C₁-C₆)alkoxy, —C═O, —(CH₂)ₙ—OH, —NH₂, NO₂, wherein n is an integer ranges from 1-10.

In preferred embodiment, the present invention relates to an improved process for the preparation of phosphate compounds of Formula (I) and pharmaceutically acceptable salts thereof.

In another embodiment, the invention provides cost-effective, viable process for the preparation of phosphate compounds of Formula (I) with high purity and good yield.

In yet another embodiment, the present invention provides improved process for the preparation of novel phosphate compounds of formula (I),

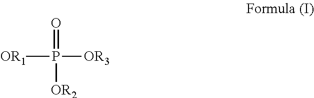

comprising the steps of:
i. preparation of free base of R₁ salt;
ii. preparation of free base of 2-(trimethylammonio)ethyl phosphate;
iii. addition of free base entity of step i) and free base 2-(trimethylammonio)ethyl phosphate in suitable solvent at ambient temperature to obtain compounds of formula-I with high yield and purity.

In another preferred embodiment, the invention provides compounds of formula I, wherein R1 is selected from the group consisting of:

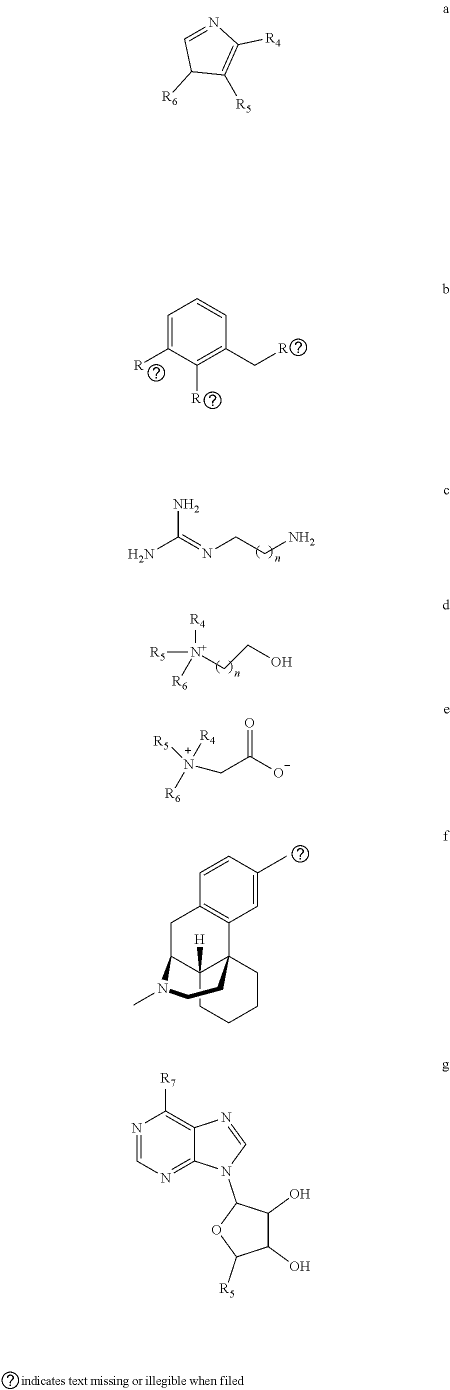

wherein R₂ is hydrogen, and R₃ is selected from

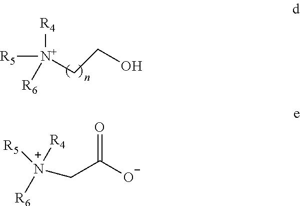

wherein R₄, R₅, R₆, R₇ and R₅ are similar or independently selected from the group consisting of hydrogen, hydroxyl, halogen, (C₁-C₆)alkyl, (C₂-C₈)alkylene, (C₂-C₈)alkyne, (C₁-C₆) alkoxy, (C₁ C₆)trifluoroalkyl, (C₃-C₈) cycloalkyl, (C₁-C₆)alkoxy, —C═O, —(CH₂)—OH, —NH₂, and NO₂.

In another preferred embodiment, the invention provides compounds of formula I, wherein R1 salt is selected from the group consisting of amino acid salts, chloride, sulfate, phosphate, diphosphate, bromide, nitrate salts, hydrochloride, hydrobromide, malate, maleate, fumarate, tartrate, succinate, citrate, taurate, threonate, acetate, lactate, methanesulfonate, benzoate, ascorbate, sulfonate, salicylate as well as, sodium, potassium, calcium, lithium, magnesium and ammonium (including substituted ammonium).

The term “solvents” as used herein, represents a compound or mixture or substance, ordinarily a liquid, in which other materials dissolve to form a solution. The role of a solvent in a chemical reaction can either be non-participatory or participatory which depends on the type of solvent and its strength. Some solvents do not participate in chemical reactions. They simply serve as the reaction medium to enable chemical reactions to occur more rapidly.

In further embodiment, the solvent used in the reaction is a polar or nonpolar or mixtures thereof wherein solvents are water, acetone, acetonitrile, dimethylformamide (DMF), dimelthylsulfoxide (DMSO), isopropanol, propanol, butanol, ethanol, and methanol; pentane, hexane, toluene, diethyl ether, benzene, THF, ethyl acetate, 1,4-Dioxane, chloroform, carbon tetrachloride, acetic acid either alone or mixture thereof.

In some embodiment, the green solvent used is lower alcohol like ethanol or methanol which can be easily recycled on completion of the reaction by distillation.

In further embodiment, the ambient temperature is maintained in the range of 20° C. to 45° C. at the time of free base addition.

In yet another preferred embodiment, the phosphate compounds of Formula I is selected from the group consisting of 1,3,4,9,10,10a-Hexahydro-6-methoxy-11-methyl-2H-10,4alpha-iminoethanophenanthren 2-(trimethylammonio)ethyl hydrogen phosphate; 4-guanidinobutan-1-aminium 2-(trimethylammonio)ethyl hydrogen phosphate; 1H-pyrrole 2-(trimethylammonio)ethyl hydrogen phosphate; 3-Hydroxymethylpyridine 2-(trimethylammonio)ethyl hydrogen phosphate; (2R,3R,4S,5R)-2-(6-amino-9H-purin-9-yl)-5-(hydroxymethyl)oxolane-3,4-diol 2-(trimethylammonio)ethyl hydrogen phosphate; 9-[(2R,3R,4S,5R)-3,4-dihydroxy-5-(hydroxymethyl)oxolan-2-yl]-6,9-dihydro-3H-purin-6-one 2-(trimethylammonio)ethyl hydrogen phosphate and like thereof.

In another embodiment, the free base of compounds of Formula I is optionally stabilized with pharmaceutically acceptable salts such as, chloride, bromide, hydrochloride, hydrobromide, hydrate, sodium, magnesium, carbonate, calcium and like thereof.

In another embodiment, the presently improved in situ process provides compounds of Formula I with high purity and yield, wherein the HPLC purity is not less than 90% after drying.

In another embodiment, the presently improved in situ process provides compounds of Formula-I with high purity and yield, wherein the yield and purity is not less than 70%.

Preferably, the practical yield is in the range of 75% to 100%.

In another embodiment, the invention provides novel phosphate comprising biochemical entities for treatment of CNS or other metabolic disorders.

Further the present novel compounds are useful in the treatment of CNS disorders such as trauma, infections, nerve degeneration, stroke, tumours, cancer, autoimmune disorders, attention deficit hyperactivity disorder, epilepsy/seizures, autism, multiple sclerosis, neuropathic pain, Alzheimer's, Huntington's disease, Parkinson's, cancer, leukaemia, bacterial skin infections, nonalcoholic steatohepatitis (NASH), fatty liver disease (FLD), alcohol related steatohepatitis, chronic glaucoma and like thereof.

Further the compounds of Formula I can be used in pharmaceutical to visualize bone abnormalities and cardiovascular abnormalities and also used as an ingredient in some products to prevent iron deficiency anemia.

The term “therapeutically effective amount” denotes an amount that reduces the risk, potential, possibility or occurrence of a disease or disorder, or provides advanced alleviation, mitigation, and/or reduction or restoration or modulation, regulation of at least one indicator/biomarker (e.g., blood or serum CRP level), and/or minimize at least one clinical symptom related to metabolic or nervous system related disorder.

The term ‘subject in need thereof’ pertains to a subject preferably mammal, more preferably a human suffering or suspected with metabolic or nervous system related disorder.

In the context of the present invention, the term “treatment” refers to alleviate, mitigate, prophylaxis, attenuate, manage, regulate, modulate, control, minimize, lessen, decrease, down regulate, up regulate, moderate, inhibit, restore, suppress, limit, block, decrease, prevent, inhibit, stabilize, ameliorate, cure, heal metabolic or nervous system related disorders observed in the patients.

Notably, the present novel phosphate compounds are non-hazardous, non-toxic, and safe for human consumption without any severe adverse effects, therefore the present medicinal composition can also be used as preventive therapy/adjuvant therapy/add-on therapy/combination/adjunctive therapy in a subject in need thereof.

Certain compounds of the present invention exist in unsolvated forms as well as solvated forms, including hydrated forms. Further, some compounds of the present invention exist in multiple crystalline or amorphous forms (“polymorphs”). Compounds of the invention are formulated in geometric or, enantiomeric or stereoisomeric forms.

In general, all physical forms are of use in the methods contemplated by the present invention and are intended to be within the scope of the invention.

Compound or pharmaceutically acceptable salts includes, hydrates, polymorphs, solvates, enantiomers or racemates. Some of the crystalline forms of the compound exist as polymorphs and as such are intended to be included in the present disclosure. In addition, some of the compounds may form solvates with water (i.e., hydrates) or common organic solvents, and such solvates are intended to be encompassed by some embodiments.

In one embodiment, the invention provides medicinal composition comprising novel phosphate compounds of Formula-I is present in effective amount along with pharmaceutically acceptable excipients.

As used herein, the term “pharmaceutically acceptable carriers, diluents or excipients” is purported to mean, without limitation, any adjuvant, carrier, excipient, sweetening agent, diluents, preservative, dye/colorant, flavor enhancer, surfactant, wetting agent, dispersing agent, suspending agent, stabilizer, isotonic agent, solvent, emulsifier, or encapsulating agent, encapsulating polymeric delivery systems or polyethyleneglycol matrix, which is acceptable for use in the subject, preferably humans. Excipients also include, for example: antiadherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, or waters of hydration, salts.

In another embodiment, the invention relates to medicinal composition of phosphate compounds of Formula-I prepared in a manner well known in the pharmaceutical art and administered by a variety of routes, depending upon whether local or systemic treatment is desired and upon the area to be treated.

The preferable route of administration includes but is not limited to sublingual, rectal, topical, parenteral, nasal, or oral.

In another embodiment, the compounds of Formula I of the present invention are non-toxic, cost effective, enriched with nutrients or biomolecules; and provide safeguard against problems associated with neurotransmission without any adverse effect.

In some embodiments, the compounds of Formula-I can be formulated in medicaments by using pharmaceutically acceptable excipients such as diluents, binders, lubricants, solubilizing agents, surfactants, stabilizers, colors, flavoring agents, sweeteners, glidants, plasticizers, and other additives.

In another embodiment of the present invention, the diluents are selected from starches, hydrolyzed starches, partially pregelatinized starches, anhydrous lactose, cellulose powder, lactose monohydrate, sugar alcohols such as sorbitol, xylitol and mannitol, silicified microcrystalline cellulose, ammonium alginate, calcium carbonate, calcium lactate, dibasic calcium phosphate (anhydrous/dibasic dehydrate/tribasic), calcium silicate, calcium sulphate, cellulose acetate, corn starch, pregelatinized starch, dextrin, β-cyclodextrin, dextrates, dextrose, erythritol, ethyl cellulose, fructose, fumaric acid, glyceryl palmitostearate, magnesium carbonate, magnesium oxide, maltodextrin, maltose, medium-chain triglycerides, polydextrose, polymethacrylates, sodium alginate, sodium chloride, sterilizable maize, sucrose, sugar spheres, talc, trehalose, xylitol, vehicles like petrolatum, dimethyl sulfoxide and mineral oil or the like.

In some embodiment of the invention, the diluent in the composition/formulation is present in a range of 1% to 30% by weight of the total composition/formulation.

In yet another embodiment of the invention, the binder is selected from disaccharides such as sucrose, lactose, polysaccharides and their derivatives like starches, cellulose, or modified cellulose such as microcrystalline cellulose and cellulose ethers such as hydroxypropyl cellulose (HPC); hydroxypropyl methyl cellulose (HPMC); sugar alcohols such as xylitol, sorbitol, or mannitol; protein like gelatin; synthetic polymers such as polyvinylpyrrolidone (PVP), polyethylene glycol (PEG), starch, acacia, agar, alginic acid, calcium carbonate, calcium 15 lactate, carbomers, carboxymethylcellulose sodium, carrageenan, cellulose acetate phthalate, chitosan, co-povidone, corn starch, pregelatinized starch, cottonseed oil, dextrates, dextrin, dextrose, ethyl cellulose, guar gum, hydrogenated vegetable oil, mineral oil, hydroxyethyl cellulose, hydroxymethyl cellulose, hydroxyl ethyl methyl cellulose, hydroxypropyl cellulose, inulin, cellulose, methyl cellulose, polyvinylpyrrolidone and polyethylene glycol, lactose, liquid 20 glucose, hypromellose, magnesium aluminum silicate, maltodextrin, maltose, methyl-cellulose, microcrystalline cellulose, pectin, poloxamer, polydextrose, polymethacrylates, povidone, sodium alginate, stearic acid, sucrose, sunflower oil, various animal vegetable oils, and white soft paraffin, paraffin, flavorants, colorants and wax.

In further embodiment of the present invention, the binder in the composition/formulation is present in a range of 0.1 to 40% by weight of the composition/formulation.

In another embodiment of the present invention, the lubricant is selected from magnesium stearate, zinc stearate, calcium stearate, glycerin monostearate, glyceryl behenate, glyceryl palmitostearate, hydrogenated castor oil, hydrogenated vegetable oil, light mineral oil, magnesium lauryl sulphate, medium-chain triglycerides, mineral oil, myristic acid, palmitic acid, poloxamer, polyethylene glycol, sodium benzoate, sodium chloride, sodium lauryl sulphate, sodium stearyl fumarate, stearic acid, talc, potassium, or sodium benzoate or the like.

In some embodiment of the present invention, the lubricant in the composition/formulation is present in a range of 0.1% to 10.0% by weight of the total composition/formulation.

In another embodiment of the present invention, the solubilizing agent is selected from polysorbate 80, sodium lauryl sulphate, anionic emulsifying wax, nonionic emulsifying wax, glyceryl monooleate, phospholipids, polyoxyethylene alkyl ethers, polyoxyethylene castor oil derivatives, polyoxyethylene sorbitan fatty acid esters, polyoxyethylene stearates, polyoxylglycerides, sorbitan esters, triethyl citrate, vitamin E, polyethylene glycol succinate, microcrystalline cellulose, carboxymethylcellulose sodium, diethanolamine, ethylene glycol palmitostearate, glycerin monostearate, hypromellose, hypromellose, acetate succinate, lecithin, polyethylene alkyl ethers, aluminum oxide, poly(methylvinyl ether/maleic anhydride), calcium carbonate, crospovidone, cyclodextrins, fructose, hydroxpropyl betadex, oleyl alcohol, povidone, benzalkonium chloride, benzethonium chloride, benzyl alcohol, benzyl benzoate, cetylpyridinium chloride, inulin, meglumine, poloxamer, pyrrolidone, sodium bicarbonate, starch, stearic acid, sulfobutylether beta cyclodextrin, tricaprylin, triolein, docusate sodium, glycine, alcohol, self-emulsifying glyceryl monooleate, cationic benzethonium chloride, cetrimide, xanthan gum, lauric acid, myristyl alcohol, butylparaben, ethylparaben, 15 methylparaben, propylparaben, sorbic acid or the like.

In another embodiment of the present invention, the amount of solubilizing agent or surfactant in the composition/formulation ranges from 0.1% to 10% by weight of the composition/formulation.

In a preferred embodiment of the present invention, the solubilizing agent or surfactant is present in a range of 0.1% to 5.0% by weight of the composition/formulation.

In some embodiment of the present invention, the glidant is selected from colloidal silicon dioxide, magnesium stearate, fumed silica (colloidal silicon dioxide), starch, talc, calcium phosphate tribasic, cellulose powdered, hydrophobic colloidal silica, magnesium oxide, zinc stearate, magnesium silicate, magnesium trisilicate, silicon dioxide or the like.

In another embodiment of the present invention, the glidant in the composition/formulation is present in a range of 0.1% to 5.0% by weight of the total composition/formulation.

In some embodiment of the present invention, the stabilizers are selected from the group consisting of alginate, agar, carrageen, gelatin, guar gum, gum arabic, locust bean gum, pectin, starch, xanthan gum, trehalose and likewise.

In some embodiment of the present invention, the stabilizer in the composition/formulation is present in a range of 0.1% to 8.0% by weight of the total composition/formulation. In some embodiment of the invention, the plasticizers are added to coating formulations selected from the group propylene glycol, glycerol, glyceryl triacetate (triacetin), triethyl citrate, acetyl triethyl citrate, diethyl phthalate, actetylated monoglycerides, castor oil, mineral oil and like thereof.

In some embodiment of the present invention, the plasticizer in the composition/formulation is present in a range of 0.1% to 5.0% by weight of the total composition/formulation.

In some embodiment of the present invention, the solvent is selected from water, alcohol, isopropyl alcohol, propylene glycol, mineral oil, benzyl alcohol, benzyl benzoate, flavored glycol, carbon dioxide, castor oil, corn oil (maize), cottonseed oil, dimethyl ether, albumin, dimethylacetamide, ethyl acetate, ethyl lactate, medium-chain triglycerides, methyl lactate, olive oil, peanut oil, polyethylene glycol, polyoxyl, castor oil, propylene carbonate, pyrrolidone, safflower oil, sesame oil, soybean oil, sunflower oil, water-miscible solvents, organic polar or non-polar solvents or mixtures thereof.

In a preferred embodiment of the present invention, the solvent in the composition/formulation is used in a quantity sufficient to make the weight of the composition/formulation 100% by weight.

The additional additives include a polymer, a plasticizer, a sweetener, and a powdered flavor, a preservative, a colorant, a surfactant, and other excipients. The powdered flavor composition includes a flavourant associated with a solid carrier. Coating materials such as synthetic polymers, shellac, corn protein (zein) or other polysaccharides, gelatin, fatty acids, waxes, shellac, plastics, and plant fibers and like thereof are used.

In a preferred embodiment of the present invention, the additives are used in a range of 1 to 20% w/w of unit dose.

In yet another embodiment, the present invention provides a medicinal composition comprising compounds of Formula I along with pharmaceutical excipients, wherein the pharmaceutical excipients are selected from a diluent, a binder, a lubricant, a glidant, an additive, a surfactant, a stabilizer or mixtures thereof.

In a preferred embodiment, the diluent is present in a range of 1 to 30%; the binder present is present in a range of 0.1 to 25%; the lubricant is present in a range of 0.1 to 10.0%; the glidant is present in a range of 0.1 to 5.0%; the additive is present in a range of 1 to 10%; the surfactant is present in a range of 0.1 to 5.0%; the stabilizer is present in a range of 0.1 to 5.0%; and the plasticizer is present in a range of 0.1 to 5.0%; by weight of total composition.

In some embodiment, the medicinal compositions of novel phosphate compounds of Formula-I are administered to a subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray.

In a further embodiment, the present composition is formulated in the form of age appropriate pediatric oral dosage forms such as syrup, inhalation, spray minitablets, chewable formulations, orodispersible films orodispersible tablets.

The magnitude of a prophylactic or therapeutic dose typically varies with the nature and severity of the condition to be treated and the route of administration. The dose, and perhaps the dose frequency, will also vary according to the age, body weight and response of the individual patient.

In general, the total daily dose (in single or divided doses) ranges from about 0.1 mg per day to about 5000 mg per day, preferably about 1 mg per day to about 1000 mg per day.

In some embodiment, the total daily dose can be administered in the range of about 1 mg to about 3000 mg per day, and preferably about 1 mg to about 1000 mg per day.

The use of any and all examples, or exemplary language (e.g., such as) provided herein, is intended merely to better illuminate the invention and does not pose a limitation on the scope of the invention unless otherwise claimed.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The invention may be further be illustrated by the following examples, which are for illustrative purposes only and should not be construed as limiting the scope of the invention in anyway.

The present disclosure is therefore to be considered as in all respects illustrative and not restrictive, the scope of the invention being indicated by the appended claims and examples, and all changes or alterations which come within the ambit of equivalency are intended to be encompassed therein.

### Examples

Having described the basic aspects of the present invention, the following non-limiting examples illustrate specific embodiments thereof. Those skilled in the art will appreciate that many modifications may be made in the invention without changing the essence of invention.

### Example 1: General Process for the Preparation of Phosphate Compounds of Formula I

### Step i)

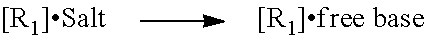

### Step ii)

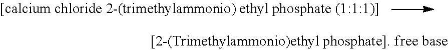

Step iii)

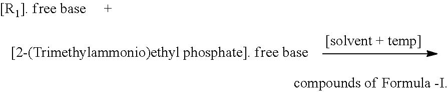

Table 1
Compounds of Formula -I

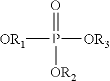

| Compound | Compound Name | R1 | R2 | R3 |
|---|---|---|---|---|
| Ia | 1,3,4,9,10,10a-Hexahydro-6-methoxy-11- methyl-2H-10,4alpha- iminoethanophenanthren 2- (trimethylammonio)ethyl hydrogen phosphate | 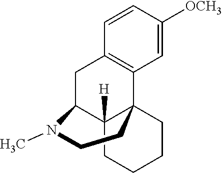 | H | 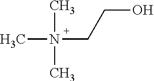 |
| Ib | 4-guanidinobutan-1-aminium 2- (trimethylammonio)ethyl hydrogen phosphate | 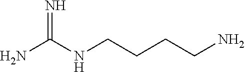 | H | 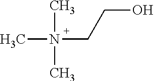 |
| Ic | 1H-pyrrole 2-(trimethylammonio)ethyl hydrogen phosphate | 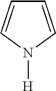 | H | 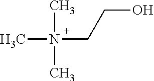 |
| Id | 3 Hydroxymethylpyridine 2- (trimethylammonio)ethyl hydrogen phosphate | 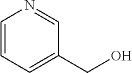 | H | 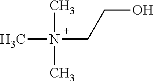 |
| Ie | (2R,3R,4S,5R)-2-(6-amino-9H-purin-9- yl)-5-(hydroxymethyl)oxolane-3,4-diol 2- (trimethylammonio)ethyl hydrogen phosphate | 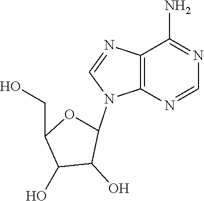 | H | 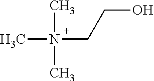 |
| If | 9-[(2R,3R,4S,5R)-3,4-dihydroxy-5- (hydroxymethyl)oxolan-2-yl]-6,9- dihydro-3H-purin-6-one 2- (trimethylammonio)ethyl hydrogen phosphate | 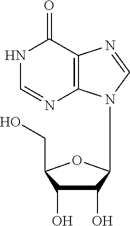 | H | 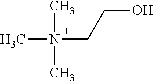 |

TABLE 2
Purity and Yield of Compounds of Formula -I

| | | % | % |
|---|---|---|---|
| Compounds | Compounds Of Formula -I | Purity | Yield |
| Ia | 1,3,4,9,10,10a-Hexahydro-6-methoxy-11-methyl- | 96 | 90 |
| | 2H-10,4alpha-iminoethanophenanthren 2- | | |
| | (trimethylammonio)ethyl hydrogen phosphate | | |
| Ib | 4-guanidinobutan-1-aminium 2- | 90 | 85 |
| | (trimethylammonio)ethyl hydrogen phosphate | | |
| Ic | 1H-pyrrole 2-(trimethylammonio)ethyl hydrogen | 93 | 76 |
| | phosphate | | |
| Id | 3 Hydroxymethylpyridine 2- | 90 | 83 |
| | (trimethylammonio)ethyl hydrogen phosphate | | |
| Ie | (2R,3R,4S,5R)-2-(6-amino-9H-purin-9-yl)-5- | 92 | 78 |
| | (hydroxymethyl) oxolane-3,4-diol 2- | | |
| | (trimethylammonio)ethyl hydrogen phosphate | | |
| If | 9-[(2R,3R,4S,5R)-3,4-dihydroxy-5- | 90 | 80 |
| | (hydroxymethyl)oxolan-2-yl]-6,9-dihydro-3H- | | |
| | purin-6-one 2-(trimethylammonio)ethyl hydrogen | | |
| | phosphate | | |

### Example 1: Process for Preparation of 1,3,4,9,10,10a-hexahydro-6-methoxy-11-methyl-2H-10,4alpha-iminoethanophenanthren 2-(trimethylammonio)ethyl hydrogen phosphate

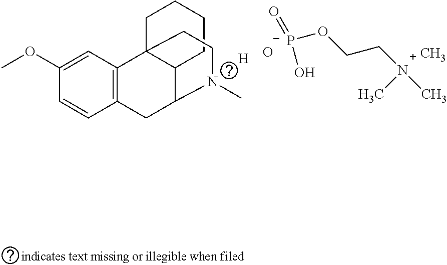

### Step i)

Water (22.6 ml) was charged in (10.0 μm) of 1,3,4,9,10,10a-hexahydro-6-methoxy-11-methyl-2H-10,4alpha-iminoethanophenanthren hydrobromide and Toluene were stirred and adjusted pH 11-13 using sodium hydroxide (4.0 μm) solution in water (16.0 ml), the reaction mass was stirred for 60 minutes to 70 minutes. The layer were separated. Aqueous layer back extracted using Toluene (10 ml). The Toluene layer was combined and wash with water (20 ml). The separated toluene layer were taken for solvent distillation under reduced pressure obtained residue was 1,3,4,9,10,10a-hexahydro-6-methoxy-11-methyl-2H-10,4alpha-iminoethanophenanthren free base (7.20 μm).

### Step ii)

(30.0 ml) water was charged and calcium chloride 2-(trimethylammonio)ethyl phosphate (1:1:1) (6.9 μm) were stirred and added calcium hydroxide (1.98 μm), The reaction mass was stirred for 60 minutes to 70 minutes. The reaction mass was filtered and collected filtrate as solution of 2-(trimethylammonio)ethyl phosphate (free base).

Step iii)

The solution of 1,3,4,9,10,10a-hexahydro-6-methoxy-11-methyl-2H-10,4alpha-iminoethanophenanthren free base was charged and added to the solution of 2-(trimethylammonio)ethyl phosphate and the reaction mass was stirred for 500 minutes to 550 minutes. The reaction mass filtered and washed with water suck dry well. Wet product were dry at 35° C. to 40° C. for 840 minutes under reduced pressure to obtain 1,3,4,9,10,10a-hexahydro-6-methoxy-11-methyl-2H-10,4alpha-iminoethanophenanthren 2-(trimethylammonio)ethyl hydrogen phosphate with yield 90%, purity 96%.

HPLC Assay-Content of 1,3,4,9,10,10a-hexahydro-6-methoxy-11-methyl-2H-10,4alpha-iminoethanophenanthren in 1,3,4,9,10,10a-hexahydro-6-methoxy-11-methyl-2H-10,4alpha-iminoethanophenanthren 2-(trimethyl ammonio)ethyl hydrogen phosphate

(On dried basis)-57.47% w/w

LOD-8.28% w/w

H1 NMR (400 MHz, DMSO) δ ppm 7.11 (1H, d), 6.81 (1H, d), 6.78 (1H, d), 4.1 (2H, t), 3.83 (3H, S), 3.71 (1H, m), 3.61 (2H t), 3.55 (1H, m), 3.30 (9H, S), 2.8 (3H S), 2.70~2.0 (8H, m), 1.63 (1H, d), 1.60~1.35 (5H, m), 1.30~1.15 (1H, m).

### Example 2: Process for Preparation of 4-guanidinobutan-1-aminium 2-(trimethylammonio)ethyl hydrogen phosphate

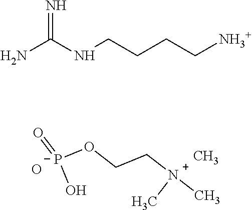

### Step i)

(30.0 ml) of water charged and 1-(4-Aminobutyl) guanidine sulfate (10.0 μm) were stirred and added Calcium Hydroxide (3.25 μm), the reaction mass was stirred for 60 minutes to 70 minutes. Further the reaction mass was filtered and collected filtrate as solution of 1-(4-Aminobutyl) guanidine free base.

### Step ii)

(30.0 ml) water was charged and calcium chloride 2-(trimethylammonio)ethyl phosphate (1:1:1) (11.29 μm) were stirred and added Calcium Hydroxide (3.08 μm), the reaction mass was stirred for 60 minutes to 70 minutes. The reaction mass was filtered and collected filtrate as solution of 2-(trimethylammonio)ethyl phosphate (free base).

Step iii)

The solution of 1-(4-Aminobutyl) guanidine free base was charged and added to the solution of 2-(trimethylammonio)ethyl phosphate and the reaction mass was stirred for 600 minutes to 720 minutes. Further the reaction mass was filtered and washed with water suck dry well. Wet product were dried at 25° C. to 30° C. for 1440 minutes under reduced pressure to obtain 4-guanidinobutan-1-aminium 2-(trimethylammonio)ethyl hydrogen phosphate with yield 85% and purity 90%.

HPLC Assay-Content of (4-Aminobutyl) guanidine in 4-guanidinobutan-1-aminium 2-(trimethylammonio)ethyl hydrogen phosphate (On dried basis)-37.28% w/w LOD-8.42% w/w

H1 NMR (400 MHz, D20) 8 ppm: 1.65-1.70 (m, 4H, 2×methylene of 1-(4-Aminobutyl) guanidine), 3.02 (t, 2H, Methylene), 3.19-3.22 (S, 9H, 3×N-Methyl, 2H methylene of 1-(4-Aminobutyl) guanidine), 3.55 (t, 2H, methylene of 2-hydroxy-N,N,N-trimethylethanaminium), 4.13 (t, 2H, methylene of 2-hydroxy-N,N,N-trimethylethanaminium).

### Example 3: Pharmaceutical Composition of Compounds of Formula I

| | Unit dose | % Unit dose |
|---|---|---|
| Ingredient | (in mg) | range |
| Formula-I a | 50 | 50%-90% |
| Magnesium Stearate | 1-5 | 0.1-10% |
| Ascorbic acid | 0.1-2 | 0.1-10% |
| Microcrystalline | 0.1-2 | 0.1-10% |
| Cellulose | | |
| Colloidal Silicon | 0.1-2 | 1-10% |
| dioxide | | |
| Hydroxypropyl | 0.1-2 | 1-10% |
| Methylcellulose | | |
| Sucrose | 0.1-5 | 1-10% |
| PVPP | 0.1-2 | 0.1-5% |
| Talc | 0.1-1 | 0.1-5% |
| Tween 80 | 0.1-2 | 0.1-5% |
| Mannitol | 0.1-2 | 0.1-5% |
| Water | QS | QS |
| Average weight | 60-70 mg | 100% |

1. Phosphate compounds of Formula I: 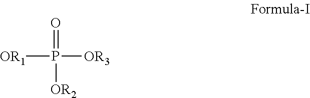
wherein R₁, R₂ and R₃ is similar or independently selected from the group consisting of hydrogen, hydroxyl, halogen, (C₁-C₆)alkyl, (C₂-C₈)alkylene, (C₂-C₈)alkyne, trifluoroalkyl, (C₃-C₈) cycloalkyl, (C₁-C₆)alkoxy, (C₆-C₁₀) aryl; heterocyclic groups; wherein the heterocyclic groups are selected from a group consisting of substituted or unsubstituted pyrrole, pyridine, indole, and quinoline;
or
polyamine, wherein the polyamine is guanidine; or quaternary ammonium; or polycyclic aromatic hydrocarbon, wherein the polycyclic aromatic hydrocarbon is selected from a group consisting of phenanthrene and anthracene; or nucleosides, wherein the nucleosides are selected from a group consisting of adenosine, guanosine, and inosine;
or
monovalent metal ion, wherein the monovalent metal ion is selected from the group consisting of Li⁺, Na⁺, and K⁺; divalent metal ion, wherein the divalent metal ion is selected from the group consisting of Ca²⁺, Mg²⁺, Zn²⁺, or combination thereof.

2. The compounds as claimed in claim 1,
wherein R₁, R₂ and R₃ are independently selected from the group consisting of hydrogen, hydroxyl, halogen, (C₁-C₆)alkyl; or group selected from 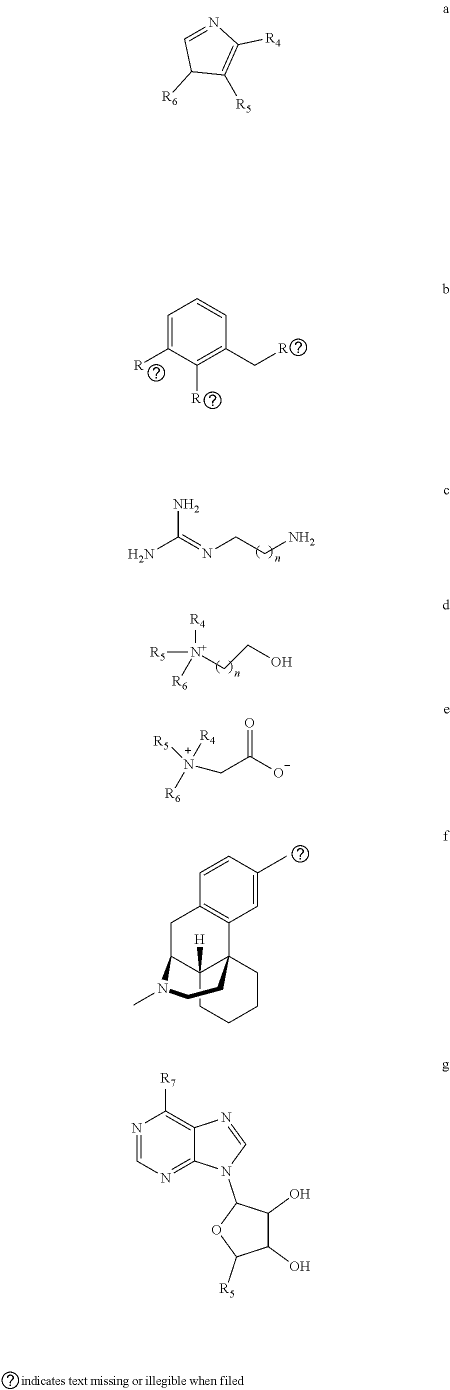
wherein R₄, R₅, R₆, R₇ and R₈ are similar or independently selected from the group consisting of hydrogen, hydroxyl, halogen, (C₁-C₆)alkyl, (C₂-C₈)alkylene, (C₂-C₈)alkyne, (C₁-C₆)alkoxy, (C₁ C₆)trifluoroalkyl, (C₃-C₈) cycloalkyl, (C₁-C₆)alkoxy, —C═O, —(CH₂)ₙ—OH, —NH₂, —NO₂; and
wherein n is an integer ranges from 1-10.

3. The compounds as claimed in claim 1, wherein the compounds is selected from the group consisting of 1,3,4,9,10,10a-Hexahydro-6-methoxy-11-methyl-2H-10,4alpha-iminoethanophenanthren 2-(trimethyl ammonio)ethyl hydrogen phosphate; 4-guanidinobutan-1-aminium 2-(trimethyl ammonio)ethyl hydrogen phosphate; 1H-pyrrole 2-(trimethyl ammonio)ethyl hydrogen phosphate; 3-Hydroxymethylpyridine 2-(trimethyl ammonio)ethyl hydrogen phosphate; (2R,3R,4S,5R)-2-(6-amino-9H-purin-9-yl)-5-(hydroxymethyl)oxolane-3,4-diol 2-(trimethyl ammonio)ethyl hydrogen phosphate; and 9-[(2R,3R,4S,5R)-3,4-dihydroxy-5-(hydroxymethyl)oxolan-2-yl]-6,9-dihydro-3H-purin-6-one 2-(trimethyl ammonio)ethyl hydrogen phosphate.

4. A process for the preparation of the phosphate compounds of Formula I, 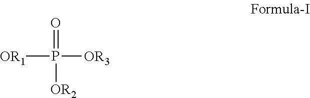
comprising steps of:
i. preparing free base of R₁ salt;
ii. preparing free base of 2-(trimethylammonio)ethyl phosphate;
iii. adding the free base of step i) and the free base of 2-(trimethylammonio)ethyl phosphate in a solvent at an ambient temperature to obtain compounds of Formula-I with high yield and purity;
wherein R₁ is selected from the group consisting of: 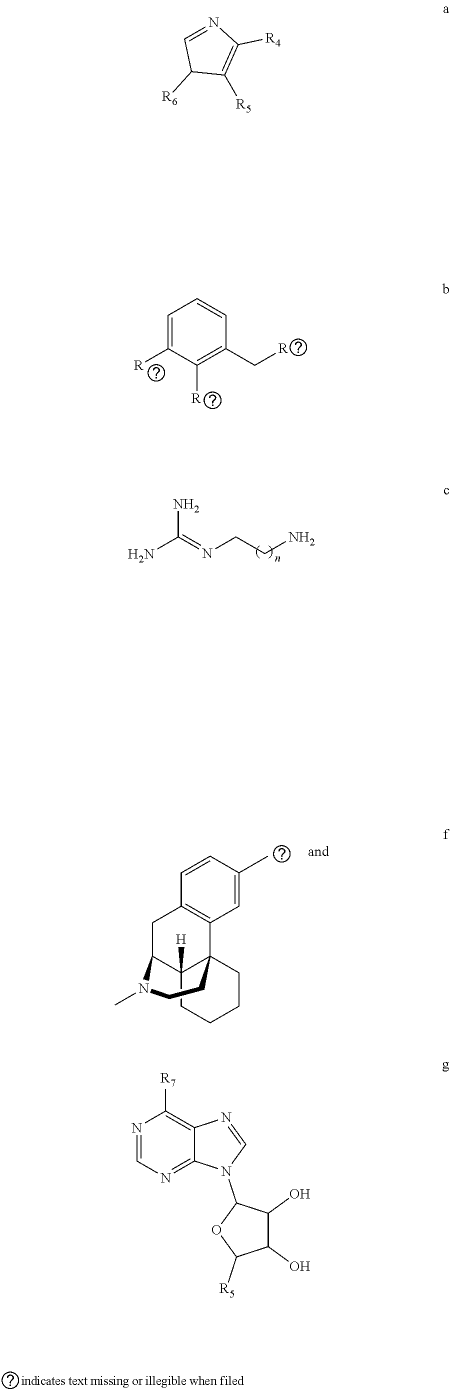
wherein R₂ is hydrogen, and R₃ is selected from 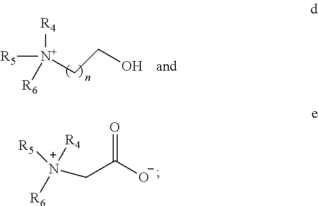
wherein R₄, R₅, R₆, R₇ and R₈ are similar or independently selected from the group consisting of hydrogen, hydroxyl, halogen, (C₁-C₆)alkyl, (C₂-C₈)alkylene, (C₂-C₈) alkyne, (C₁-C₆)alkoxy, (C₁ C₆)trifluoroalkyl, (C₃-C₈) cycloalkyl, (C₁-C₆)alkoxy, —C═O, —(CH₂)—OH, —NH₂, and NO₂.

5. The process as claimed in claim 4, wherein R₁ salt is selected from the group consisting of amino acid salts, chloride, sulfate, phosphate, diphosphate, bromide, nitrate salts, hydrochloride, hydrobromide, malate, maleate, fumarate, tartrate, succinate, citrate, taurate, threonate, acetate, lactate, methanesulfonate, benzoate, ascorbate, sulfonate, salicylate, sodium, potassium, calcium, lithium, magnesium, ammonium, (and substituted ammonium).

6. The process as claimed in claim 4, wherein the solvent is selected from water, acetone, acetonitrile, dimethylformamide (DMF), dimelthylsulfoxide (DMSO), isopropanol, propanol, butanol, ethanol, and methanol; pentane, hexane, toluene, diethyl ether, benzene, THF, ethyl acetate, 1,4-dioxane, chloroform, carbon tetrachloride, and acetic acid, and a mixture of any of the foregoing.

7. The process as claimed in claim 4, wherein the ambient temperature is in the range of 20° C. to 45° C.

8. The process as claimed in claim 4, wherein the compounds of Formula I are selected from the group consisting of 1,3,4,9,10,10a-Hexahydro-6-methoxy-11-methyl-2H-10,4alpha-iminoethanophenanthren 2-(trimethyl ammonio)ethyl hydrogen phosphate; 4-guanidinobutan-1-aminium 2-(trimethyl ammonio)ethyl hydrogen phosphate; 1H-pyrrole 2-(trimethyl ammonio)ethyl hydrogen phosphate; 3-Hydroxymethylpyridine 2-(trimethyl ammonio)ethyl hydrogen phosphate; (2R,3R,4S,5R)-2-(6-amino-9H-purin-9-yl)-5-(hydroxymethyl)oxolane-3,4-diol 2-(trimethyl ammonio)ethyl hydrogen phosphate; and 9-[(2R,3R,4S,5R)-3,4-dihydroxy-5-(hydroxymethyl)oxolan-2-yl]-6,9-dihydro-3H-purin-6-one 2-(trimethyl ammonio)ethyl hydrogen phosphate.

9. A composition comprising a compound of Formula-I as claimed in claim 1 and at least one pharmaceutically acceptable excipient.

10. The composition as claimed in claim 9, wherein the pharmaceutically acceptable excipient is selected from the group consisting of diluent, binder, lubricant, glidant, additive, surfactant, stabilizer and plasticizer; wherein the diluent is present in a range of 1 to 30 wt %; wherein the binder present is present in a range of 0.1 to 25 wt %; wherein the lubricant is present in a range of 0.1 to 10.0 wt %; wherein the glidant is present in a range of 0.1 to 5.0 wt %; wherein the additive is present in a range of 1 to 10 wt %; wherein the surfactant is present in a range of 0.1 to 5.0 wt %; wherein the stabilizer is present in a range of 0.1 to 5.0 wt %; and wherein the plasticizer is present in a range of 0.1 to 5.0 wt % by weight of the composition.

11. A method of treating a CNS disorder in a patient comprising administering a therapeutically effective amount of a compound of claim 1 to the patient.
